(19) United States
(12) Patent Application Publication
(10) Pub. No.: US 2025/0032167 A1
(43) Pub. Date: Jan. 30, 2025

(54) HAND-HELD SURGICAL DEVICE AND INSULATING INSERT FOR A HAND-HELD SURGICAL DEVICE

(71) Applicant: OLYMPUS WINTER & IBE GMBH, Hamburg (DE)

(72) Inventors: Christoph Knopf, Stockelsdorf (DE); Christian Brockmann, Hollenstedt (DE)

(73) Assignee: OLYMPUS WINTER & IBE GMBH, Hamburg (DE)

(21) Appl. No.: 18/675,731

(22) Filed: May 28, 2024

Related U.S. Application Data
(60) Provisional application No. 63/528,992, filed on Jul. 26, 2023.

(51) Int. Cl. A61B 18/14; A61B 18/00

(52) U.S. Cl. CPC A61B 18/14; A61B 2018/00083; A61B 2018/00184; A61B 2018/00505; A61B 2018/00601; A61B 2018/00625; A61B 2018/1407

(57) ABSTRACT

An insulating insert for a hand-held surgical device, by means of which insulating insert a human body is protected or not traumatized during use of the hand-held device. This is achieved by the fact that an insulating insert for a hand-held surgical device is formed like a tube and, at a proximal end region, has coupling means for detachable coupling to a distal end of a tubular shaft of the hand-held device. A circumference of the tube-like insulating insert has at least one flexible component. Through this flexible component on the insulating insert, mechanical forces between the urethra and the hand-held device can be damped during the insertion of the device into the human body.

The invention relates to an insulating insert for a hand-held surgical device according to the preamble of claim 1. The invention further relates to a hand-held surgical device according to claim 13.

Hand-held surgical devices, in particular resectoscopes, of the type in question are used mainly in electrosurgical procedures in urology. These devices are customarily used for resection and vaporization of tissue, e.g. of tissue in the lower urinary tract. To this end, the hand-held device, in particular the resectoscope, can have a longitudinally displaceable electrosurgical passage instrument which, after the insertion of the device into the body to be treated, can be advanced with its distal working end out of a distal end of a shaft tube of the hand-held device. The electrosurgical passage instrument or the electrode instrument has an electrosurgical electrode at its distal end. This electrode can, for example, have the form of a loop or of a button or the like.

For the abovementioned uses, a radiofrequency electric current is applied to the electrode. In doing this, it should be ensured that the electrode does not come into electrical contact with the shaft tube, in particular an outer shaft tube or outer tube, of the hand-held device. If such electrical contact were to take place, a short circuit could cause a defect in the device and lead to unforeseeable damage in the body that is to be treated.

In order to avoid short circuits of this kind, the hand-held devices or resectoscopes usually comprise, at their distal end region, a portion that is made of an electrically insulating material. This portion is conventionally referred to as an insulating tip or also as an insulating insert. The insulating insert can be fastened either to an inner shaft or shaft tube, in which an electrode carrier is guided, or to the outer shaft or outer tube. Since these hand-held devices or the resectoscopes are designed for repeated use and therefore have to be regularly sterilized or autoclaved, the insulating insert has to satisfy strict requirements as regards durability and re-usability. The choice of material is therefore generally restricted to relatively expensive high-performance ceramics, for example silicon nitrite. Electrically insulating plastics are used as an alternative to these ceramics, at least for single-use articles. These plastics are in particular thermally stable, so that they do not melt immediately upon contact with the electrode.

The aforementioned materials all behave in a mechanically rigid way. This property is particularly problematic when inserting the instrument into the urethra. As soon as the insulating insert or part of the insulating insert comes into contact with the urethra, a force, which is applied to insert the instrument into the body and for all other relative movements between instrument and body, is transmitted directly opposite this contact region to the urethra or the mucosa. In particular, small contact surfaces lead to a high mechanical stress in the mucosa. This can inevitably lead to trauma or damage to the mucosa.

The problem addressed by the invention is therefore to make available an insulating insert and a hand-held surgical device through which the body is protected or not traumatized during use of the instrument.

A solution to this problem is described by the features of claim 1. Accordingly, it is provided that an insulating insert for a hand-held surgical device, in particular for a resectoscope, is formed like a tube and, at a proximal end region, has coupling means for detachable coupling to a distal end of a tubular shaft of the hand-held device. A key feature of the invention is that a circumference of the tube-like insulating insert has at least one flexible component. Through this flexible component on the insulating insert, mechanical forces between the urethra and the hand-held instrument can be damped during the insertion of the instrument into the human body. The mechanical force or stress spreads over a larger contact surface with the mucosa and leads to a deformation of the flexible component. This can prevent or at least reduce trauma to the urethra.

It is preferably provided that the at least one flexible component is arranged on the circumference of a distal end region of the insulating insert. According to this exemplary embodiment, the insulating insert comes to bear with the flexible component directly on the inside of the urethra or on the mucosa, as a result of which the force can be distributed more evenly over the contact surface. The positioning of the flexible component at the distal end region or the distal end face of the insulating insert is particularly advantageous for the production of the insulating insert. In addition, this positioning of the flexible component allows the force to be absorbed particularly efficiently.

In particular, the invention provides that the flexible component is produced from a silicone or an elastomer. The insulating insert can be produced from a rigid material, in particular a ceramic or a plastic. Through this choice of material, the insulating insert meets the requirements described above; at the same time, the combination with the flexible material can solve the stated problem and allows the instrument to be moved in the body in a particularly gentle way. When choosing the material of the flexible component, it is important for reusable instruments that the flexible component is particularly heat-resistant and can be processed from time to time.

In a preferred exemplary embodiment of the invention, provision can be made that the flexible component extends in a ring shape around the entire circumference of the distal end region. In this exemplary embodiment, the component can be considered as a ring-like flexible extension of the insulating insert. With the ring-like design of the flexible component, it is possible to ensure that, each time the insulating insert comes into contact with the mucosa, mechanical forces are absorbed and trauma thus prevented. The orientation of the instrument or of the insulating insert relative to the urethra makes no difference. This offers the surgeon maximum flexibility in working.

Furthermore, it is conceivable that the flexible component has a hardness of 10 Shore A to 90 Shore A, preferably 30 Shore A to 70 Shore A. It has been shown that this hardness is particularly suitable for the described use.

In a further alternative exemplary embodiment of the invention, provision can be made that the flexible component extends over an angular range of 45° to 180°, in particular over an angular range of 90°, on the entire circumference of the distal end region. In particular, if the distal end region of the insulating insert does not lie in a plane, but is formed for example like a beak, it may be advantageous if only a most distally oriented portion is provided with the flexible component. Depending on the design of the insulating insert, the flexible component can extend over a defined angular range on the distal circumference.

Preferably, it is conceivable that the flexible component is arranged on the outer side or top side and/or the inner side and/or the front side of the distal end region, and/or the flexible component extends on the outer side in the direction of the proximal end. Depending on the structure of the hand-held surgical device and the intended use, it may be advantageous that the component extends over different areas of the distal end region of the insulating insert. The further the flexible component extends in the distal direction, the greater the force-absorbing effect. According to the invention, it is conceivable that the flexible component has in the axial direction a length of 0.3 mm to 7.0 mm, preferably 0.5 mm to 2.0 mm.

In particular, it is conceivable that two, three or more flexible components are arranged on the circumference. For uses in which there is the risk of trauma to the urethra and in which a free edge of the distal region of the insulating insert is to be used, provision can be made that a plurality of individual flexible components are arranged along the circumference.

In addition, in a particularly preferred exemplary embodiment of the invention, provision can be made that the distal end region of the insulating insert has an axial and/or radial or ring-like undercut for improved adherence of the flexible component. In the production of the insulating insert, the component is poured over the distal region. The undercut provides the flexible component with anchoring that is strong enough to prevent the flexible component from coming loose under the effect of radial or axial forces. The undercut increases the contact surface between the flexible component and the insulating insert. This increase in the contact surface also serves to stabilize the connection between the flexible component and the insulating insert. Depending on the structure or embodiment of the insulating insert, it may be advantageous to align the undercut axially, i.e. parallel to a longitudinal axis of the insulating insert, or radially, i.e. along the circumference. As an alternative to the undercut, it is likewise possible that the distal end region of the insulating insert has holes or notches or other depressions in which the flexible component can settle.

Finally, it is also conceivable that the flexible component and a jacket surface of at least the distal region of the insulating insert form a continuous surface. This design with a continuous surface can prevent the insulating insert from catching within the urethra or can prevent difficulties from arising in terms of achieving sufficient reprocessing for the reuse of the insulating insert.

A hand-held surgical device for solving the stated problem has the features of claim 13. In this case, the hand-held surgical device can in particular be a resectoscope or the like. The claimed hand-held surgical device has an inner shaft or shaft tube and an outer shaft or outer tube. The outer tube is arranged around the inner shaft. According to the invention, an insulating insert according to claim 1 is detachably assigned to a distal end of the inner shaft. By this arrangement of the insulating insert at the distal end of both the inner shaft and the outer shaft, it is possible to prevent a situation where an electrosurgical tool or an electrode, which is led through the inner shaft out of the insulating insert, comes into electrical contact with the metallic shaft tubes.

A preferred exemplary embodiment of the invention is described in more detail below with reference to the drawing, in which:

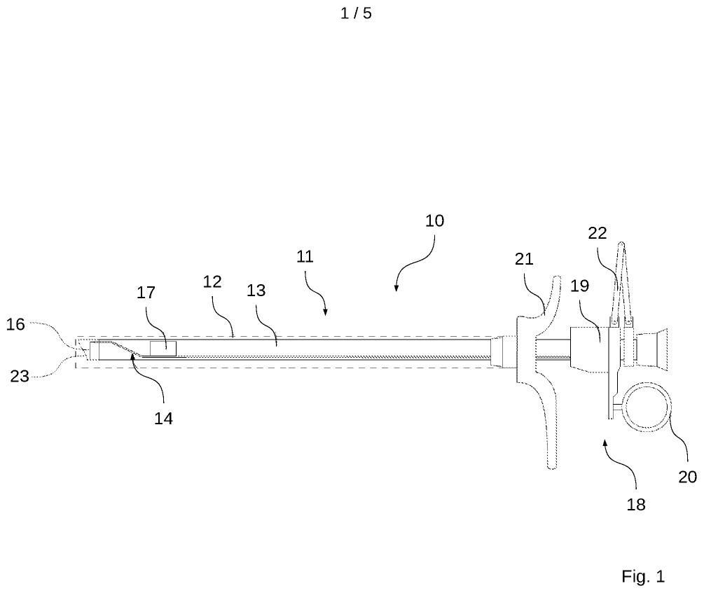

FIG. 1 shows a schematic view of a hand-held surgical device, in particular a resectoscope,

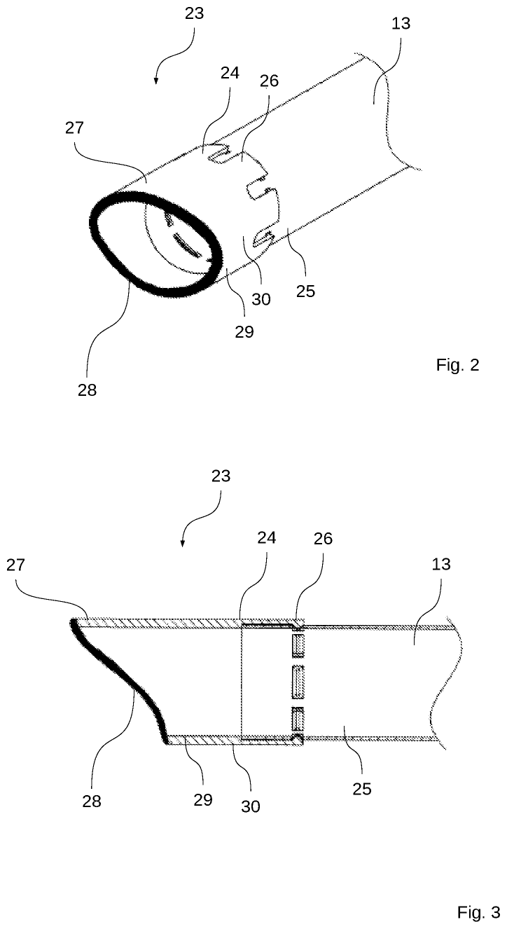

FIG. 2 shows a perspective view of an insulating insert,

FIG. 3 shows a sectional view of the insulating insert according to FIG. 2,

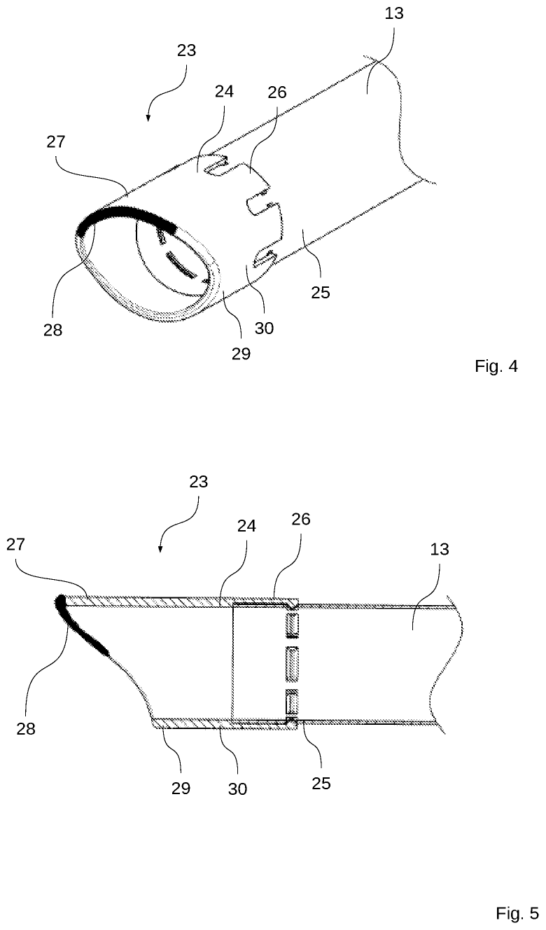

FIG. 4 shows a perspective view of a further exemplary embodiment of an insulating insert,

FIG. 5 shows a sectional view of the insulating insert according to FIG. 4,

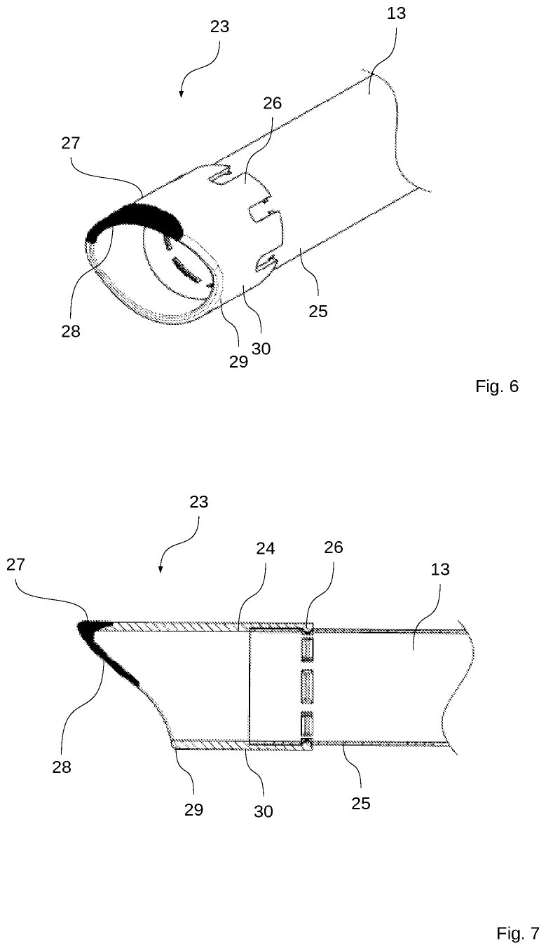

FIG. 6 shows a perspective view of a further exemplary embodiment of an insulating insert,

FIG. 7 shows a sectional view of the insulating insert according to FIG. 6, and

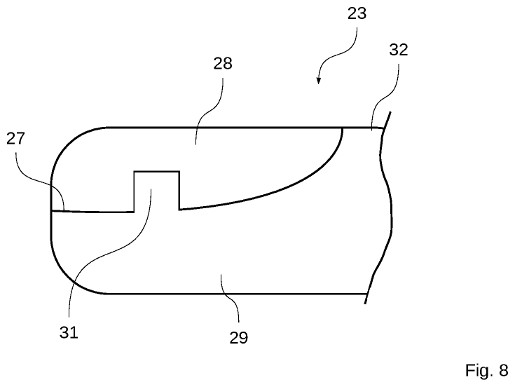

FIG. 8 shows a sectional view through a distal end region of the insulating insert.

FIG. 1 shows a schematic cross-sectional side view of a resectoscope 10. It is expressly noted that this resectoscope 10 is shown simply as one possible exemplary embodiment of the claimed hand-held surgical device. It is equally conceivable that the hand-held surgical device is another instrument different than the resectoscope 10 shown here.

The resectoscope 10 has a resectoscope shaft 11, which comprises an outer shaft 12 or outer tube. A tubular inner shaft 13 runs inside the outer shaft 12. An electrode instrument 14 and an optical unit 15 are shown inside the inner shaft 13. In addition, further elements not shown here can run inside the resectoscope 10, for example a separate irrigation tube and the like.

The electrode instrument 14 has, at a distal end, an electrosurgical tool or an electrode 16. The electrode 16 shown here is formed as a loop, but it can also be configured as a button or the like. It will be seen from FIG. 1 that the electrode instrument 14 is protected by a retention element 17, with a partially circular cross section, against displacements, i.e. displacements deviating from the longitudinal direction of the resectoscope shaft 11. The electrode instrument 14 is mounted longitudinally displaceably in the inner shaft 13. The retention element 17 is of a shape complementary to the inner wall of the inner shaft 13 or to the outer wall of the optical unit 15 and has a partially cylindrical shape.

By actuation of a handle 18, the electrode instrument 14 can be moved in a constrained axial movement in the distal and proximal direction. It can be pushed out beyond the distal end of the inner shaft 13 and of the outer shaft 12. The operator is thus also able to manipulate tissue that is located further away from the resectoscope tip. For this purpose, the inner shaft 13 and/or the electrode instrument 14 can also be mounted rotatably about their longitudinal axis. For the manipulation of the tissue, a radiofrequency electric current is applied to the electrode 16.

The resectoscope 10 shown in FIG. 1 has a passive transporter in which, by relative movement of the grip parts 20 and 21 arranged proximally on the resectoscope shaft 11, a carriage 19 is displaced in the distal direction toward the distal, first grip part 21 counter to a spring force applied by a spring bridge 22. In the displacement of the carriage 19 in the distal direction toward the grip part 21, the electrode instrument 14 is displaced in the distal direction in a manner not shown. Upon relaxation of the grip parts 20, 21, the spring force generated by the spring bridge 22 forces the carriage 19 back to its starting position, wherein the electrode instrument 14 is pulled in the proximal direction. Upon the return displacement of the carriage 19, an electrosurgical intervention can be performed with the electrode 16 without manual force from the operator, i. e. passively.

An insulating insert 23 is detachably coupled with its proximal end 24 to the distal end 25 of the inner shaft 13 (see FIG. 2, for example, in which only a distal region of the device is shown). For this purpose, it is provided that the insulating insert 23 has coupling means 26 at its proximal end 24. For the treatment of the patient, the resectoscope 10, with the insulating insert 23 to the front, is pushed into the patient's urethra. In order to prevent the aforementioned trauma to the urethra or the mucosa, a circumference 27 of the insulating insert 23 has a flexible component 28. As is illustrated in the exemplary embodiment according to FIGS. 2 and 3, this flexible component 28 can be assigned to the circumference 27 of the distal end region 29 of the insulating insert 23. In the exemplary embodiment shown, the flexible component 28 can be considered as a kind of ring. According to the invention, this flexible component 28 is produced from a silicone, an elastomer or another flexible material. If, during the insertion of the resectoscope 10, the insulating insert 23 with the flexible component 28 to the front now makes contact with the mucosa, no injury or trauma is caused, but rather an elastic deformation of the flexible component 28 takes place. Thus, the transmitted mechanical stress does not lead to deformation and ultimately to injury of the mucosa. The insulating insert 23 is produced from a rigid material, such as a ceramic, a composite, a plastic or the like, so that the shape of the insulating insert 23 does not in itself change, and the functionality of the resectoscope 10 is not impaired.

In the exemplary embodiment shown in FIGS. 2 and 3, the entire distal end region 29 of the insulating insert 23 is surrounded by the flexible component 28, so that the mucous membrane of the urethra is protected independently of the contact surface of the insulating insert 23.

Alternatively, it is equally conceivable that only a sub-region of the distal end region 29 of the insulating insert 23 has a flexible component 28. Thus, for example in FIGS. 4 and 5, an exemplary embodiment is shown in which only an upper region of the circumference 27 is provided with the flexible component 28. It will be seen that the flexible component 28 extends over the inner side, the front side and the outer side of the distal end region 29. The upper region of the circumference 27 is thus at least partially enclosed by the flexible component 28.

In FIGS. 6 and 7, an exemplary embodiment is shown in which this enclosing of the upper region of the circumference 27 is even more pronounced. Here, the flexible component 28 extends on the top side or jacket surface 30 of the insulating insert 23 in the direction of the proximal end 24. Through this increase in size of the flexible component 28, a greater amount of force applied from the outside to the insulating insert 23 or to the mucosa can be absorbed.

According to the invention, the flexible component 28 has a width of 0.3 mm to 7.0 mm, preferably 0.5 mm to 2.0 mm. In this case, the component 28 is dimensioned such that it lies in a plane with the jacket surface 30 of the insulating insert 23, so that there is no constriction between the outer shaft 12 and the inner shaft 13.

For a particularly stable and firm connection between the flexible component 28 and the circumference 27 of the insulating insert 23, provision can be made that the circumference 27 has an undercut 31. As is shown in FIG. 8 for example, the circumference 27 of the distal end region 29 can be assigned a radial undercut 31 having a cuboid cross section. This undercut 31 lends additional mechanical resistance to the flexible component 28, which is for example injected around the distal end region 29, and, in the event of an external force being applied, protects against unwanted detachment of the flexible component 28 from the insulating insert 23. FIG. 8 shows only a detail of a sectional view of a wall 32 of the insulating insert 23. Alternatively or additionally, it is also conceivable that the distal end region 29 is assigned an axial undercut or a plurality of smaller radial undercuts, in order thereby to increase the stability of the connection between the flexible component 28 and the insulating insert 23.

### LIST OF REFERENCE SIGNS

10 resectoscope
11 resectoscope shaft
12 outer shaft
13 inner shaft
14 electrode instrument
15 optical unit
16 electrode
17 retention element
18 handle
19 carriage
20 grip part
21 grip part
22 spring bridge
23 insulating insert
24 proximal end
25 distal end
26 coupling means
27 circumference
28 flexible component
29 distal end region
30 jacket surface
31 undercut
32 wall

1. An insulating insert for a hand-held surgical device,
in particular for a resectoscope, wherein the tube-like insulating insert has, at a proximal end region, coupling means for detachable coupling to a distal end of a tubular shaft of the hand-held device, wherein a circumference of the tube-like insulating insert has at least one flexible component.

2. The insulating insert as claimed in claim 1, wherein the at least one flexible component is arranged on the circumference of a distal end region of the insulating insert.

3. The insulating insert as claimed in claim 1, wherein the flexible component is produced from silicone or an elastomer, and/or the insulating insert from a rigid material.

4. The insulating insert as claimed in claim 1, wherein the flexible component extends in a ring shape around the entire circumference of the distal end region.

5. The insulating insert as claimed in claim 1, wherein the flexible component has a hardness of 10 Shore A to 90 Shore A.

6. The insulating insert as claimed in claim 1, wherein the flexible component extends over an angular range of 45° to 180° on the entire circumference of the distal end region.

7. The insulating insert as claimed in claim 1, wherein the flexible component is arranged on a portion of the circumference of the distal end region that reaches furthest in the distal direction.

8. The insulating insert as claimed in claim 1, wherein the flexible component is arranged on the outer side or top side and/or the inner side and/or the front side of the distal end region, and/or the flexible component extends on the outer side in the direction of the proximal end.

9. The insulating insert as claimed in claim 1, wherein two, three or more flexible components are arranged on the circumference.

10. The insulating insert as claimed in claim 1, wherein the flexible component has a length in the axial direction of 0.3 mm to 7.0 mm.

11. The insulating insert as claimed in claim 1, wherein the distal end region of the insulating insert has an axial and/or radial or ring-like undercut for improved adherence of the flexible component.

12. The insulating insert as claimed in claim 1, wherein the flexible component and a jacket surface of at least the distal region of the insulating insert form a continuous surface.

13. A hand-held surgical device with an inner shaft or shaft tube and with an outer shaft or outer tube, and with an insulating insert as claimed in claim 1, wherein the insulating insert is able to be coupled detachably to the inner shaft, and the outer shaft is movable over the inner shaft and the insulating insert.
